(19) United States
(12) Patent Application Publication
(10) Pub. No.: US 2023/0256199 A1
(43) Pub. Date: Aug. 17, 2023

(54) Catheter Insertion System

(71) Applicant: Bard Access Systems, Inc., Salt Lake City, UT (US)

(72) Inventor: Glade H. Howell, Draper, UT (US)

(21) Appl. No.: 18/109,103

(22) Filed: Feb. 13, 2023

Related U.S. Application Data
(60) Provisional application No. 63/310,515, filed on Feb. 15, 2022.

(51) Int. Cl. A61M 25/00

(52) U.S. Cl. CPC A61M 25/0097; A61M 25/0026

(57) ABSTRACT

A catheter assembly including a catheter and a stylet disposed within a lumen of the catheter, the lumen having a closed distal end. The stylet includes a sensor at the distal end of the stylet and the sensor is located within a sensor pocket at the closed distal end of the lumen. An aperture extending through a luminal wall between the sensor pocket and the exterior of the catheter defines an electrical pathway between the sensor and the patient vasculature. A tapered distal portion of the catheter extends away from the distal end of the catheter and the sensor pocket is disposed along the tapered distal portion. A fluid opening extending between the lumen and the exterior of the catheter is disposed proximal the sensor pocket. A coated portion of the stylet includes an electrically insulative coating and the coated portion is disposed adjacent the fluid opening.

### PRIORITY

This application claims the benefit of priority to U.S. Provisional Application No. 63/310,515, filed Feb. 15, 2022, which is incorporated by reference in its entirety into this application.

### BACKGROUND

Proper placement of a central venous catheter (“CVC”) is important to function and longevity of the catheter. Current methods for placing the tip of the CVC include tracking both electrocardiograph changes in the P-wave and the tip of the CVC in three-dimensional space as the CVC is inserted within the vena cava. A stylet having a magnetic region is inserted into the CVC catheter to provide rigidity to the catheter during insertion and allows the magnetic region to be tracked using magnetic field detection. However, there is a distance between the magnetic region of the stylet and the tip of the CVC, leading to an approximation of the tip of the CVC during placement. It would be beneficial to the clinician and the patient to have a catheter insertion system wherein the electrocardiograph changes in the P-wave are more quickly detected and the tip of the stylet is located closer to the tip of the CVC, allowing the tip of the CVC to be more accurately placed within the pericardial vena cava. Disclosed herein is a catheter insertion system, a method of use, and a method of manufacturing that address the foregoing.

### SUMMARY

Disclosed herein is a catheter assembly that, according to some embodiments, includes a catheter configured for placement within a vasculature of a patient, where the catheter includes (i) a catheter tube configured for placement within a vasculature of a patient, the catheter tube defining a distal end; (ii) a first lumen extending along the catheter tube, where the first lumen is in fluid communication with a first extension leg; and (iii) a second lumen extending along the catheter tube, where the second lumen in fluid communication with a second extension leg. The catheter assembly further includes a stylet disposed within the second lumen, where the stylet includes a sensor configured to detect an electrical signal emanating from the patient. The sensor is located at the distal end of the stylet. The stylet is positioned with the second lumen such that the sensor is disposed within a sensor pocket of the second lumen, where the sensor pocket is positioned at a closed distal end of the second lumen, and where an aperture extending through a luminal wall of the second lumen defines a fluid pathway between the sensor pocket and the vasculature.

In some embodiments, the aperture defines an electrical pathway between sensor and the vasculature.

In some embodiments, the aperture is positioned adjacent the pocket. In some embodiments, the second lumen includes a fluid opening extending through the luminal wall, and the fluid opening is disposed proximal the aperture.

In some embodiments, the catheter tube includes a tapered distal portion extending proximally away from the distal end of the catheter tube, where the tapered distal portion defines a first diameter at the distal of the catheter tube end and a second diameter at a proximal end of the tapered distal portion, where the second diameter is greater than the first diameter, and further where the pocket is disposed along the tapered distal portion.

In some embodiments, the distance between the distal end and the pocket is less than one half of a length of the tapered distal portion. In some embodiments, the distance between the distal end and the pocket is less than 2 cm.

In some embodiments, the stylet includes a coating extending along a coated portion of the stylet within the second lumen, where the coating is electrically insulative.

In some embodiments, the fluid opening is disposed adjacent the coated portion. In some embodiments, the coated portion extends along an entire length of the stylet disposed within the second lumen. In some embodiments, the coating substantially occupies an annular space between the stylet and the lumina wall. In some embodiments, the coating includes a lubricant.

Also disclosed herein is a method of placing a catheter within a vasculature that, according to some embodiments, includes (i) inserting a stylet into a lumen of the catheter, where the lumen has a closed distal end; (ii) advancing the stylet along the lumen such that a sensor of the stylet is disposed within a sensor pocket of the lumen, where the sensor pocket is disposed at the closed distal end, and where an aperture defining a fluid pathway extends through a luminal wall between the sensor pocket and an exterior of the catheter. The method further includes advancing the catheter along the vasculature and discontinuing advancement in response to an electrical signal detected by the sensor, where the aperture defines an electrical pathway for the electrical signal.

In some embodiments, the method further includes visually observing through the aperture the stylet disposed within the sensor pocket.

In some embodiments of the method, the aperture defines an electrical pathway between sensor and vasculature.

In some embodiments of the method, the catheter includes a tapered distal portion extending proximally away from a distal end of the catheter, where the tapered distal portion defines a first diameter at the distal end and a second diameter at a proximal end of the tapered distal portion. In such embodiments, the second diameter is greater than the first diameter, and the aperture is disposed along the tapered distal portion.

In some embodiments of the method, the distance between the distal end and the aperture is less than one half of a length of the tapered distal portion. In some embodiments of the method, the distance between the distal end and the aperture is less than 2 cm.

In some embodiments, the method further includes removing the stylet from the lumen.

Also disclosed herein is a method of manufacturing a catheter assembly that, according to some embodiments, includes (i) forming a catheter tube having a tapered distal portion extending proximally away from a distal end of the catheter tube; (ii) forming a lumen extending along the catheter tube, where the lumen has a closed distal end; (iii) forming an aperture extending through a luminal wall between the closed distal end and an exterior of the catheter tube; and (iv) inserting a stylet into the lumen such that a sensor at a distal end of the stylet is disposed at the closed distal end.

In some embodiments of the manufacturing method the aperture is disposed along the tapered distal portion. In some embodiments of the manufacturing method, a distance between the distal end and the aperture is less than one half of a length of the tapered distal portion. In some embodiments of the manufacturing method, the distance between the distal end and the aperture is less than 2 cm.

In some embodiments, the manufacturing method further includes forming a fluid opening extending through the luminal wall between the lumen and the exterior of the catheter tube, where the fluid opening is disposed proximal the tapered distal portion.

In some embodiments, the manufacturing method further includes coating the stylet with an insulative coating to define a coated portion of the stylet, where the coated portion is disposed adjacent the fluid opening.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a catheter assembly including a catheter and a stylet, in accordance with some embodiments;

FIG. 2A illustrates a cross-sectional side view of a distal portion of the catheter including the sensor pocket, in accordance with some embodiments;

FIG. 2B illustrates the cross-sectional side view of FIG. 2A further including the stylet disposed within the lumen, in accordance with some embodiments;

FIGS. 3A-3B illustrate a cross-sectional views of a distal portion of catheter assembly depicting an exemplary method of placing the stylet within the lumen, in accordance with some embodiments;

FIG. 4 illustrates a flow chart of an exemplary method of placing a catheter assembly within a vasculature, in accordance with some embodiments;

FIG. 5 illustrates an exploded view of the catheter assembly, in accordance with some embodiments; and

FIG. 6 illustrates a flow chart of an exemplary method of manufacturing the catheter assembly, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

The phrases “connected to,” “coupled to/with,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

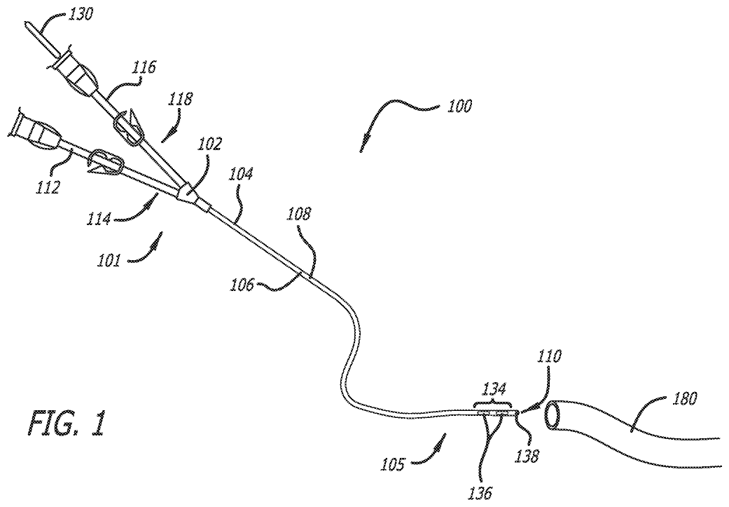

FIG. 1 illustrates a perspective view of a catheter assembly 100, in accordance with some embodiments. The catheter assembly 100 includes a catheter 101. In some embodiments, the catheter 101 includes a central venous catheter (“CVC”) having a hub 102 coupled to a catheter tube 104 extending to a distal end 110. In the illustrated embodiment, the catheter 101 includes a first lumen 106 and a second lumen 108. In other embodiments, the catheter 101 may include more than two lumens. The hub 102 is coupled to a first extension leg 112 and a second extension leg 116. The first extension leg 112 includes a first extension leg lumen 114 in fluid communication with the first lumen 106 and the second extension leg 116 includes a second extension leg lumen 118 in fluid communication with the second lumen 108. The catheter assembly 100 further includes a stylet 130 inserted into the second extension leg lumen 118 and the second lumen 108. The stylet 130 may be configured to provide rigidity to the catheter 101 to assist in placement of the catheter 101 within the vasculature.

The stylet 130 includes a sensor 138 (e.g., an electrode) configured for detecting an electrical signal within the patient body such as an ECG signal including a p-wave, for example. The sensor 138 may be disposed immediately adjacent a distal end of the stylet 130 as further described below. The sensor 138 is generally configured to enable accurate placement of a distal tip 110 of the catheter tube 104. For example, a medical device tracking system may be configured to determine the location of the sensor 138 within the vasculature 180, e.g., with the superior vena cava, based on a detected P-wave signal. As the sensor 138 is located immediately adjacent the distal end of the stylet 130, and as the distal end of the stylet 130 may be located adjacent the distal end 110 of the catheter 101, the medical device tracking system may determine the location of the distal end 110 within the vasculature 180. In some embodiments, the stylet 130 may further include a magnetic region 134 disposed adjacent the distal end of the stylet 130, where the magnetic region 134 includes a number (e.g., one, two, three or more) magnets 136 configured to generate one or more magnetic fields. In such embodiment, the medical device tracking system may be configured to track the location of the magnetic region 134 in three-dimension space based on the one or more magnetic fields.

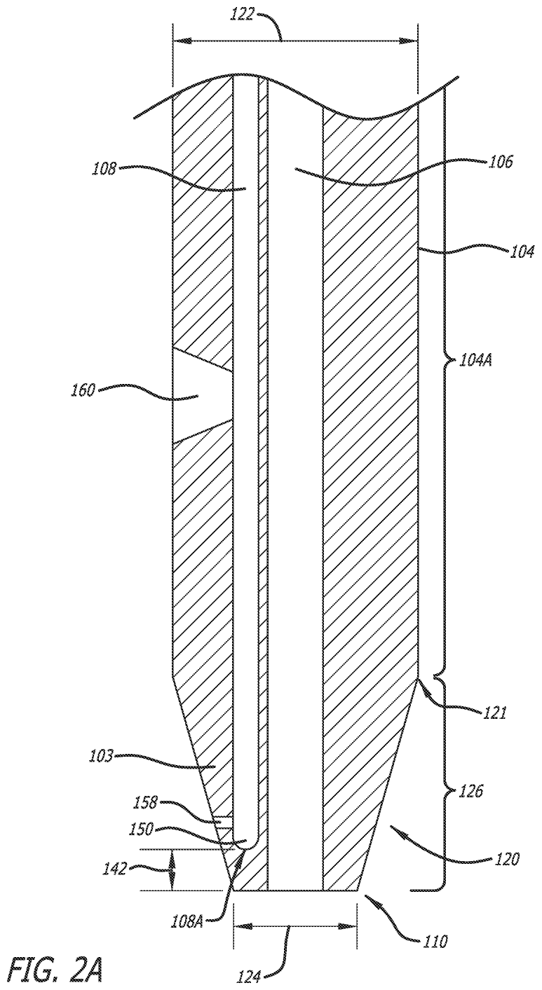

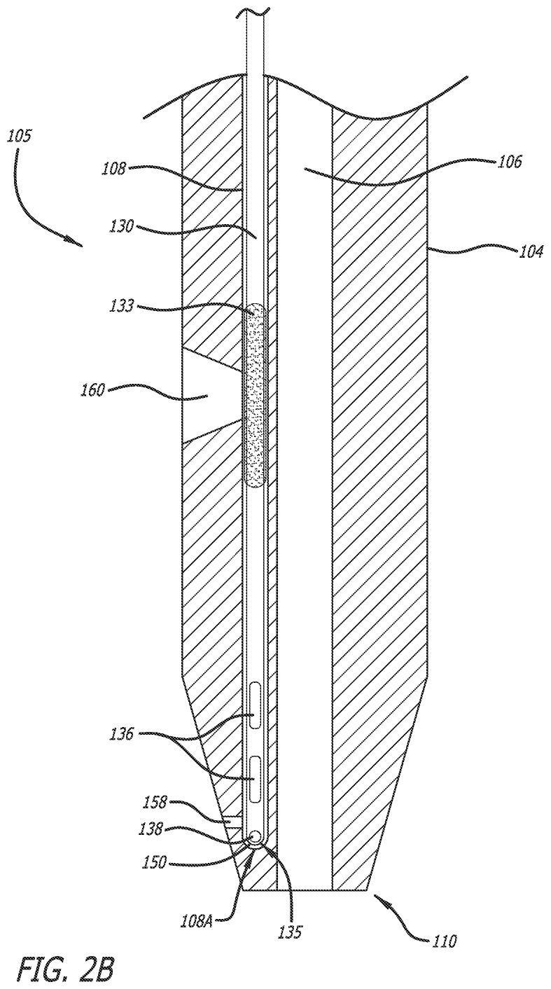

FIG. 2A illustrates a cross-sectional side view of the distal end portion 105 of the catheter tube 104, in accordance with some embodiments. Shown are the first lumen 106 and the second lumen 108. The catheter tube 104 generally defines an elongate portion 104A extending proximally away from a transition point 121 and a tapered distal portion 120 extending distally away from the transion point 121. The elongate portion 104A defines a substantially constant outside diameter 122, and the tapered distal tip portion 120 defines a connical (or tapered) shape. The tapered distal tip portion 120 includes the outside diameter 122 at the transition point 121 and a tip diameter 124 at the distal end 110, where the tip diameter 124 is less than the outside diameter 122. The transition point 121 is located a transition point distance 126 from the distal end 110 as such, the transition point distance 126 defines a length of the tapered distal tip portion 120.

The second lumen 108 extends along the distal end portion 105 and defines a closed distal end 108A located a distance 142 away from the distal end 110. In the illustrated embodiment, the distance 142 may be less than about 2 cm, 1.5 cm, 1.0 cm or 0.5 cm. In some embodiments, a distance between the distal end 110 and the closed distal end 108A is less than one half of the length of the tapered distal portion 120. A aperture 158 extends through the wall 103 of the catheter tube 104 such that, in use, the second lumen 108 is in fluid communication with the blood within the vasculature 80. The aperture 158 is disposed adjacent the closed distal end 108A. The closed distal end 108A may define a sensor pocket 150 configured to receive the distal end of the stylet 130 and the aperture 158 defines fluid communication between the sensor pocket 150 and the blood during use.

In the illustrated embodiment, the catheter tube 104 may include a side opening 160 extending through the wall 103. Such a side opening 160 may be configured for delivery of medication or any other use associated with the catheter 101. The side opening 160 may be disposed proximal the aperture 158 and/or the tapered distal tip portion 120.

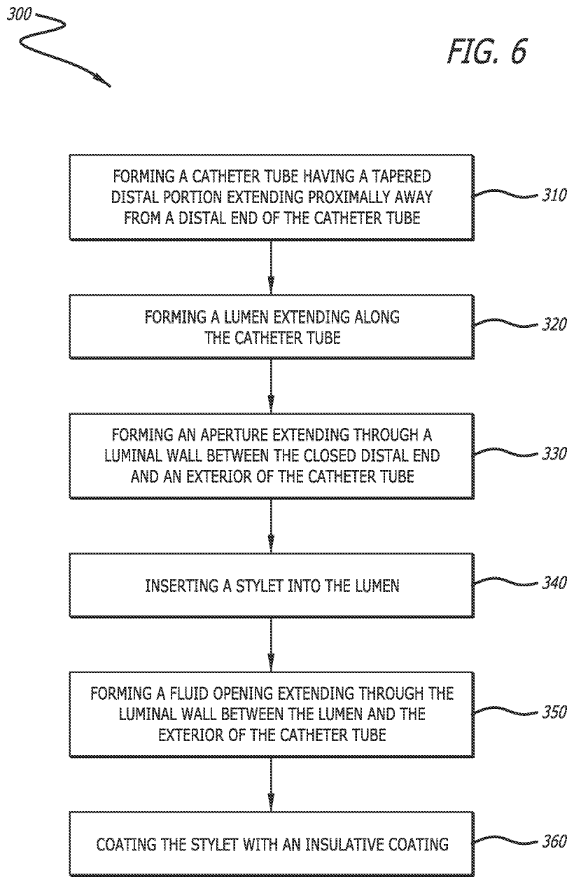

FIG. 2B illustrates the cross-sectional view of the distal end portion 105 of FIG. 6 having the stylet 130 disposed within the second lumen 108, in accordance with some embodiments. In some embodiments, the second lumen 108 may be configured to slidably receive the stylet 130 so that the distal end 135 of the stylet 130 is disposed adjacent the closed distal end 108A, i.e. within the sensor pocket 150. As the sensor 138 is disposed adjacent the distal end 135 of the stylet 130, the sensor 138 is disposed within the sensor pocket 150. Furthermore, during use, the sensor 138 is in fluid communication with the blood by way of the aperture 158, and since the blood is conductive, the sensor 138 is electrically coupled with the blood.

In some embodiments, the stylet 130 may include a coating 133 that is electrically insulative. In some embodiments, the coating 133 may extend along a portion of the stylet 130 adjacent the side opening 160 to electrically isolate the sensor 138 from the blood via the side opening 160. In some embodiments, the coating 133 extend along a substantial entirety of the stylet 130. In some embodiments, the coating 133 may include a lubrication. In some embodiments, the coating 133 may substantially occupy the annular space between stylet 130 and second lumen 108.

By way of summary, the catheter assembly 100 includes a stylet 130 having a sensor 138 disposed within the second lumen 108 of the catheter tube 104 so that the sensor 138 is disposed adjacent the distal end 110 of the catheter tube 104. During placement of the catheter 101 within the vasculature 180, the sensor 138 is in electrical communication with the blood via the aperture 158 disposed adjacent the distal end 110 so that the sensor 138 may detect an electrical signal (e.g., a p-wave). An insulative coating 133 is disposed along the stylet 130 to prevent electrical communication between the blood and the sensor 138 via an electrical pathway other than the aperture 158.

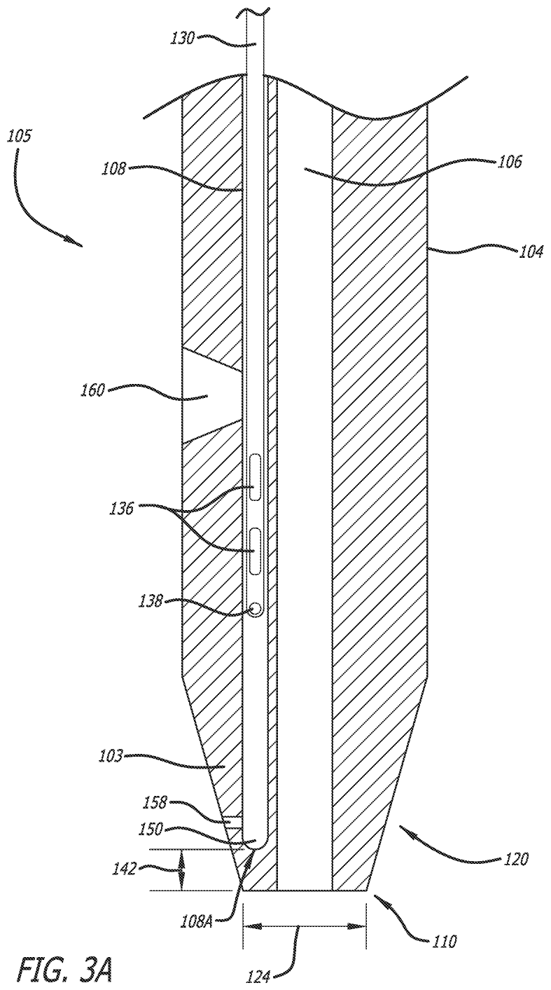

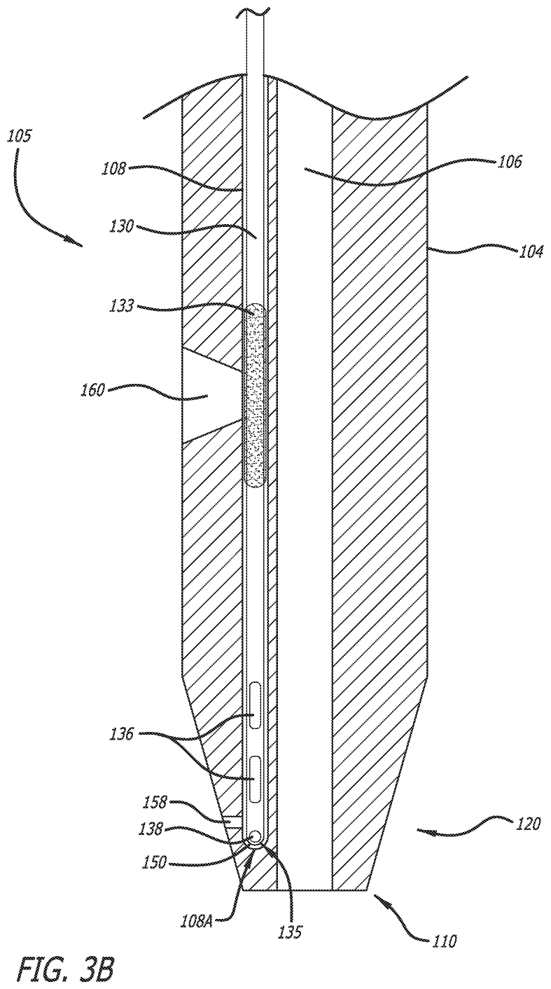

FIGS. 3A-3B are cross-sectional views illustrating an exemplary method of placing the stylet 130 within the second lumen 108 of the catheter 101, in accordance with some embodiments. FIG. 3A illustrates the stylet 130 partially inserted within the second lumen 108. As illustrated in FIG. 3A, the stylet 130 may be slidably inserted into the second lumen 108 of the catheter 101.

FIG. 3B illustrates the stylet 130 fully inserted within the second lumen 108. As illustrated in FIG. 3B, the stylet 130 may be disposed within the sensor pocket 150 prior to placing the catheter 101 within the vasculature 180. In some embodiments, the user may visually determine that the stylet 130 is disposed within the sensor pocket 150 by visually observing the stylet 130 through the aperture 158. Once the stylet 130 is within the sensor pocket 150, the stylet 130 may be rotated to orient the sensor 138 with the aperture 158 to enhance the ability of the sensor 138 to detect the P-wave through the aperture 158.

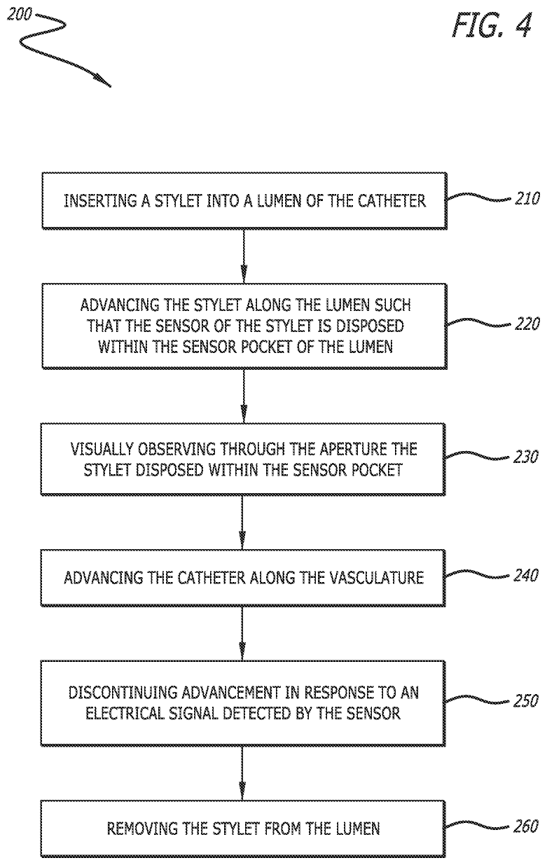

FIG. 4 illustrates a flow chart of an exemplary method 200 of placing a catheter within a vasculature that, according to some embodiments, may include all or any subset of the following actions, steps, or processes. The method 200 may include inserting the stylet into a lumen of the catheter (block 210), where the lumen has a closed distal end.

The method 200 may further include advancing the stylet along the lumen such that the sensor of the stylet is disposed within the sensor pocket of the lumen (block 220). The sensor pocket is disposed at the closed distal end of the lumen, and the aperture defines a fluid pathway extending through the luminal wall between the sensor pocket and an exterior of the catheter. In some embodiments of the method 200, the catheter includes a tapered tip distal portion extending proximally away from a distal end of the catheter, where the tapered distal tip portion defines a first diameter at the distal end and a second diameter at a proximal end of the tapered distal tip portion. In such embodiments, the second diameter is greater than the first diameter, and the aperture is disposed along the tapered distal tip portion. In some embodiments of the method 200, the distance between the distal end and the aperture is less than one half of a length of the tapered distal portion. In some embodiments of the method 200, the distance between the distal end and the aperture is less than 2 cm.

The method 200 may further include visually observing through the aperture the stylet disposed within the sensor pocket (block 230). The method 200 may further include advancing the catheter along the vasculature (240). The method 200 may further include discontinuing advancement in response to an electrical signal detected by the sensor (block 250) where the aperture defines an electrical pathway for the electrical signal. The method 200 may further include removing the stylet from the lumen (block 260).

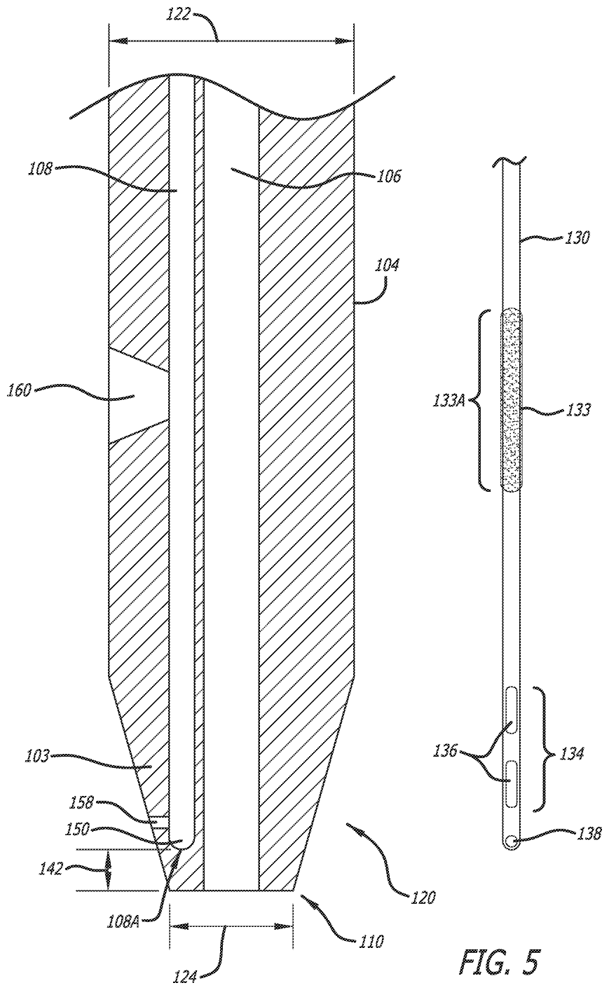

FIG. 5 illustrates an exploded view of the catheter assembly 100 with the distal end portion 105 shown in cross section, in accordance with some embodiments. In some embodiments, a method of manufacture may be used to manufacture the catheter 101 having the sensor pocket 150 therein and the stylet 130 having the sensor 138 and/or the magnetic region 134. The catheter tube 104 including the first and second lumens 106/108 may be formed, where the second lumen 108 has the closed distal end 108A. In some embodiments, the catheter tube 104 may be formed by extrusion, 3D-printing, injection molding, or the like. The catheter tube 104 includes the first catheter tube diameter 122 and the second catheter tube diameter 124. The catheter tube 104 may includes the tapered distal tip portion 120 tapering from the first catheter tube diameter 122 to the second catheter tube diameter 124.

The sensor pocket 150 may be formed at the closed distal end 108A of the second lumen 108. In some embodiments, the second lumen 108 including the sensor pocket 150 may be extruded, 3D-printing, injection molding, or the like. In the illustrated embodiment, the closed distal end 108A and the associated sensor pocket 150 may be located along the tapered distal tip portion 120.

Once the sensor pocket 150 is formed into the catheter tube 104, the aperture 158 may be formed. The aperture 158 may provide fluid communication between the sensor pocket 150 and an exterior of the catheter tube 104. In some embodiments, the aperture 158 may be formed along the tapered distal tip portion 120. In some embodiments, the aperture 158 may be drilled through, cut through the outside surface into the sensor pocket 150 or the like.

The opening 160 may be formed. In some embodiments, the opening 160 may be simultaneously formed with the aperture 158 or may be formed after the aperture 158. In some embodiments, the opening 160 may be formed proximal the aperture 158. In some embodiments, the opening 160 may be formed proximal the tapered distal tip portion 120.

Once the catheter tube 104 has been fully formed with the sensor pocket 150, the aperture 158, and the opening 160, the catheter 101 may be assembled. The catheter tube 104 may be coupled to the hub 102 and placed in fluid communication with the first and second extension legs 112/116 (see FIG. 1).

The stylet 130 may be formed. In some embodiments, the stylet 130 may include the magnets 136 coupled to the stylet 130 to define the magnetic region 134. The sensor 138 is coupled to the stylet 130. In some embodiments, the stylet 130 may include a stylet length so as to extend between the closed distal end 108A of the second lumen 108 and the proximal end of the second extension leg 116. The coating 133 may be applied to the stylet 130 along a coated region 133A of the stylet 130.

FIG. 6 illustrates a flow chart of an exemplary method 300 of manufacturing the catheter assembly 100 that, according to some embodiments, may include all or any subset of the following actions, steps, or processes. The method 300 may include forming a catheter tube having a tapered distal portion extending proximally away from a distal end of the catheter tube (block 310).

The method 300 may further include forming a lumen extending along the catheter tube (block 320), where the lumen has a closed distal end. The method 300 may further include forming an aperture extending through a luminal wall between the closed distal end and an exterior of the catheter tube (block 330). In some embodiments of the method 300, the aperture is disposed along the tapered distal tip portion. In some embodiments of the method 300, a distance between the distal end of the catheter tube and the aperture is less than one half of a length of the tapered distal tip portion. In some embodiments of the method 300, the distance between the distal end and the aperture is less than 2 cm.

The method 300 may further include inserting a stylet into the lumen (340) such that the sensor at the distal end of the stylet is disposed at the closed distal end.

The method 300 may further include forming a fluid opening extending through the luminal wall between the lumen and the exterior of the catheter tube (350), where the fluid opening is disposed proximal the tapered distal tip portion.

The method 300 may further include coating the stylet with an insulative coating (block 360) to define a coated portion of the stylet, where the coated portion is disposed adjacent the fluid opening.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter assembly, comprising:
a catheter including:
a catheter tube configured for placement within a vasculature of a patient, the catheter tube defining a distal end;
a first lumen extending along the catheter tube, the first lumen in fluid communication with a first extension leg; and
a second lumen extending along the catheter tube, the second lumen in fluid communication with a second extension leg; and
a stylet disposed within the second lumen, the stylet including a sensor configured to detect an electrical signal emanating from the patient,
wherein:
the sensor is located at the distal end of the stylet,
the sensor is disposed within a sensor pocket of the second lumen,
the sensor pocket positioned at a closed distal end of the second lumen, and
an aperture extending through a luminal wall of the second lumen defines a fluid pathway between the sensor pocket and the vasculature.

2. The assembly according to claim 1, wherein the aperture defines an electrical pathway between the sensor and the vasculature.

3. The assembly according to claim 1, wherein the aperture is positioned adjacent the pocket.

4. The assembly according to claim 1, wherein:
the catheter tube includes a tapered distal portion extending proximally away from the distal end of the catheter tube, the tapered distal portion defining a first diameter at the distal end and a second diameter at a proximal end of the tapered distal portion,
the second diameter is greater than the first diameter, and
the pocket is disposed along the tapered distal portion.

5. The assembly according to claim 4, wherein the distance between the distal end of the catheter tube and the pocket is less than one half of a length of the tapered distal portion.

6. The assembly according to claim 4, wherein the distance between the distal end of the catheter tube and the pocket is less than 2 cm.

7. The assembly according to claim 1, wherein:
the stylet includes a coating extending along a coated portion of the stylet within the second lumen, and
the coating is electrically insulative.

8. The assembly according to claim 1, wherein:
the second lumen includes a fluid opening extending through the luminal wall, and
fluid opening is disposed proximal the aperture.

9. The assembly according to claim 8, wherein the coated portion is disposed adjacent the fluid opening.

10. The assembly according to claim 8, wherein the coated portion extends along an entire length of the stylet disposed within the second lumen.

11. The assembly according to claim 8, wherein the coating substantially occupies an annular space between the stylet and the lumina wall.

12. The assembly according to claim 8, wherein the coating includes a lubricant.

13. A method of placing a catheter within a vasculature, comprising:
inserting a stylet into a lumen of the catheter, the lumen having a closed distal end;
advancing the stylet along the lumen such that a sensor of the stylet is disposed within a sensor pocket of the lumen, wherein:
the sensor pocket is disposed at the closed distal end, and
an aperture defining a fluid pathway extends through a luminal wall between the sensor pocket and an exterior of the catheter;
advancing the catheter along the vasculature; and
discontinuing advancement in response to an electrical signal detected by the sensor,
wherein the aperture defines an electrical pathway for the electrical signal.

14. The method according to claim 13, further comprising visually observing through the aperture the stylet disposed within the sensor pocket.

15. The method according to claim 13, wherein:
the catheter includes a tapered distal portion extending proximally away from a distal end of the catheter, the tapered distal portion defining a first diameter at the distal end and a second diameter at a proximal end of the tapered distal portion,
the second diameter is greater than the first diameter, and
the aperture is disposed along the tapered distal portion.

16. The method according to claim 15, wherein the distance between the distal end and the aperture is less than one half of a length of the tapered distal portion.

17. The method according to claim 15, wherein the distance between the distal end and the aperture is less than 2 cm.

18. The method according to claim 13, further comprising removing the stylet from the lumen.

19. A method of manufacturing a catheter assembly, comprising:
forming a catheter tube having a tapered distal portion extending proximally away from a distal end of the catheter tube, wherein:
the tapered distal portion extends proximally away from the distal end of the catheter tube, and
the tapered distal portion defines a first diameter at the distal end of the catheter tube and a second diameter at a proximal end of the tapered distal portion, the second diameter greater than the first diameter;
forming a lumen extending along the catheter tube, the lumen having a closed distal end;
forming an aperture extending through a luminal wall between the closed distal end and an exterior of the catheter tube; and
inserting a stylet into the lumen such that a sensor at a distal end of the stylet is disposed at the closed distal end.

20. The method according to claim 19, wherein the aperture is disposed along the tapered distal portion.

21. The method according to claim 20, wherein a distance between the distal end of the catheter tube and the aperture is less than one half of a length of the tapered distal portion.

22. The method according to claim 20, wherein the distance between the distal end of the catheter tube and the aperture is less than 2 cm.

23. The method according to claim 19, further comprising forming a fluid opening extending through a luminal wall between the lumen and the exterior of the catheter tube, the fluid opening disposed proximal the tapered distal portion.

24. The method according to claim 19, further comprising coating the stylet with an insulative coating to define a coated portion of the stylet, wherein the coated portion is disposed adjacent the fluid opening.
